(19) United States
(12) Patent Application Publication
(10) Pub. No.: US 2026/0284070 A1
(43) Pub. Date: Sep. 24, 2026

(54) Ophthalmic Solution of Brinzolamide

(71) Applicant: Micro Labs Limited, Bangalore (IN)

(72) Inventors: Rajesh Suresh Kshirsagar, Bangalore (IN); Sanjay P. Boldhane, Bangalore (IN); Ashwin B. Kuchekar, Pune (IN); Thahera Parveen Dandu

(73) Assignee: Micro Labs Limited, Bangalore (IN)

(21) Appl. No.: 19/479,042

(22) PCT Filed: May 15, 2024
(86) PCT No.: PCT/IN2024/050562

§ 371 Date: Oct. 27, 2025

(30) Foreign Application Priority Data
May 16, 2023 (IN) 202341034214

(51) Int. Cl. A61K 31/542; A61K 9/00; A61K 9/10; A61K 47/34; A61K 47/69

(52) U.S. Cl. CPC A61K 31/542; A61K 9/0048; A61K 9/10; A61K 47/34; A61K 47/6907

(57) ABSTRACT

The present invention relates to an ophthalmic composition of Brinzolamide. More particularly, the present invention relates to an ophthalmic solution of Brinzolamide comprising Brinzolamide dispersed uniformly in a polymer in form of polymeric micelles. Further, the present invention relates to aqueous micelle solutions of Brinzolamide which provide a sustained Intra Ocular Pressure lowering effect. Further, the present invention relates to aqueous micelle solutions of Brinzolamide which are more bioavailable, efficacious and also provide a sustained Intra Ocular Pressure (IOP) lowering effect than, the currently marketed ophthalmic suspensions of Brinzolamide. The present invention also relates to stable ophthalmic aqueous micelle solutions of Brinzolamide.

### FIELD OF THE INVENTION

The present invention relates to an ophthalmic composition of Brinzolamide. More particularly, the present invention relates to an ophthalmic solution of Brinzolamide comprising Brinzolamide dispersed uniformly in a polymer in form of polymeric micelles. Further, the present invention relates to aqueous micelle solutions of Brinzolamide which provide a sustained Intra Ocular Pressure lowering effect. Further, the present invention relates to aqueous micelle solutions of Brinzolamide which are more bioavailable, efficacious and also provide a sustained Intra Ocular Pressure (IOP) lowering effect than, the currently marketed ophthalmic suspensions of Brinzolamide. The present invention also relates to stable ophthalmic aqueous micelle solutions of Brinzolamide.

### BACKGROUND OF THE INVENTION

Brinzolamide is designated chemically as (R)-(+)-4-Ethylamino-2-(3-methoxypropyl)-3,4-dihydro-2H-thieno[3, 2-e]-1, 2-thiazine-6-sulfonamide-1,1-dioxide. The chemical structure of Brinzolamide is represented by the following formula:

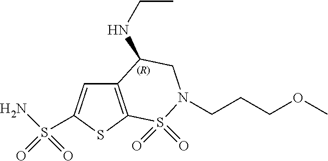

It is indicated for the treatment of elevated intraocular pressure in patients with ocular hypertension or open-angle glaucoma.

Brinzolamide is disclosed in U.S. Pat. Nos. 5,240,923 and 5,378,703.

It has very limited solubility at physiological pH. It is plausible that, due to limited solubility of Brinzolamide between pH 6.0 to 7.5, the clinical trials for its ophthalmic preparations were carried out using suspensions of Brinzolamide rather than a solution of Brinzolamide.

Elevated intraocular pressure is a major risk factor in the pathogenesis of optic nerve damage and glaucomatous visual field loss. Brinzolamide is an inhibitor of carbonic anhydrase II (CA-II). Following topical ocular administration, Brinzolamide inhibits aqueous humor formation and reduces elevated intraocular pressure. Currently, a conventional standard 1% w/v suspension of Brinzolamide (Azopt®) is used in the treatment of elevated intraocular pressure (IOP) in patients with ocular hypertension or open-angle glaucoma. Azopt® contains a polymer carbomer 974P and tyloxapol as a surfactant to solubilize Brinzolamide. Each mL of Azopt® (Brinzolamide ophthalmic suspension, 1% contains) Brinzolamide 10 mg and inactive ingredients include 0.1 mg benzalkonium chloride as a preservative, edetate disodium as a preservative and chelating agent, sodium chloride and mannitol as tonicity agents, tyloxapol as a wetting agent (surfactant) and carbomer 974P as a suspending agent; and sodium hydroxide or hydrochloric acid for pH adjustment to approximately 7.5. Azopt® is supplied in natural, plastic DROP-TAINER® dispensers with a controlled dispensing tip containing 5 mL. It has to be stored at 4° C. to 30° C. and is suitable for multi-dose topical ophthalmic use.

The recommended dose of Azopt® as per USFDA approved label, is one drop in the affected eye(s) three times daily.

WO9825620 A1 discloses that conventional sterilization methods like autoclaving cannot be employed in the manufacture of suspensions comprising Brinzolamide since the compound recrystallizes as large needle-shaped crystals, after autoclaving and subsequent cooling.

EP0941094 A1 discloses Ophthalmic suspensions containing brinzolamide or brinzolamide and a beta-blocker and processes for manufacturing the suspensions. As per the disclosure, the use of Tyloxapol or Triton X-100 avoids crystallization of brinzolamide after autoclaving.

The suspension dosage form for ophthalmic administration has its own disadvantages such as low bioavailability, inconvenience to the patient, problems with physical stability like sedimentation and compaction, etc.

Various attempts have been made to provide Brinzolamide in the form of ophthalmic solutions.

US20160339105 A1 discloses sterile aqueous formulation of Brinzolamide in combination with polymers like Soluplus® i.e., polyvinyl caprolactam-polyvinyl acetate-polyethylene glycol graft co-polymer which need a surfactant like polysorbate 80 for solubilizing Brinzolamide.

US20210353635 A1 discloses an aqueous solution comprising brinzolamide, solubilizing agents, isotonizing agents, chelating agent, viscosity enhancing polymer, buffering agent and purified water. The solution comprises at least 0.5 w/v % Brinzolamide dissolved in the solution, hydroxy-propyl-β-cyclodextrin; polyvinyl caprolactam-polyvinyl acetate-polyethylene glycol graft copolymer; and water. The document teaches the use of cyclodextrin class of compounds for solubilizing Brinzolamide.

There is an unmet need for improved Brinzolamide ophthalmic solution, with a higher efficacy than the ophthalmic suspensions of Brinzolamide. It would be preferred, to avoid or eliminate use of excipients like solubilizing agents or surfactants to solubilize Brinzolamide. It would be most preferable, to use solubilized Brinzolamide that can be conveniently formulated into aqueous solutions.

After extensive experimentation, the inventors of the present invention have come up with improved ophthalmic aqueous solutions with solubilized Brinzolamide. The Brinzolamide is present in the form of nano-sized polymeric micelles in the solution. The solutions have been prepared by minimal use of excipients and have no need for additional surfactants or solubilizing agents to solubilize Brinzolamide. Additionally, the aqueous micelle solutions provided have sustained Intra Ocular Pressure lowering effect. There are also provided aqueous micelle solutions of Brinzolamide having higher efficacy than the Brinzolamide suspensions of similar strength. The solutions are stable to accelerated stability temperature and humidity conditions.

### OBJECTIVES OF THE INVENTION

An objective of the invention is to provide an ophthalmic solution composition of Brinzolamide. Further objective of the invention is to provide an aqueous micelle solution comprising Brinzolamide dispersed uniformly in a polymer; in the form of polymeric micelles; wherein the polymer in the micelle is a polyvinyl caprolactam-polyvinyl acetate-polyethylene glycol graft co-polymer; and wherein the solution does not contain a surfactant.

### SUMMARY OF THE INVENTION

The present invention provides an ophthalmic solution of Brinzolamide.

In an embodiment, the present invention provides an aqueous micelle solution comprising Brinzolamide dispersed uniformly in a polymer; in the form of polymeric micelles; wherein the polymer in the micelle is a polyvinyl caprolactam-polyvinyl acetate-polyethylene glycol graft co-polymer, and wherein the solution does not contain a surfactant.

In a preferred aspect of the above embodiment, there is provided the ophthalmic solution wherein, the solution further does not contain a solubilizer of the cyclodextrin class of compounds. In a most preferred aspect of the embodiment, there is provided the ophthalmic solution wherein, the solution further does not contain any solubilizer other than polyvinyl caprolactam-polyvinyl acetate-polyethylene glycol graft co-polymer.

In an alternative embodiment, the present invention provides an aqueous micelle solution comprising 1% w/v Brinzolamide in the form of polymeric micelles, wherein the Brinzolamide is dispersed uniformly in a polymer, wherein the polymer in the micelle is a polyvinyl caprolactam-polyvinyl acetate-polyethylene glycol graft co-polymer. Wherein the solution does not contain a surfactant and exhibits higher Tmax and AUC values as compared to conventional standard 1% w/v suspension of Brinzolamide, when the solution and the suspension are administered in equal volume, to a mammal by instillation in the eye of the mammal. In a preferred aspect of this embodiment, the Tmax is reached between 3.5 to 4.5 hours after instillation in the eye of the mammal. In another preferred aspect of this embodiment, the AUC is about 117 h mm Hg. In another preferred aspect of this embodiment, IOP reduction after instillation of the solution in the eye of the mammal, is in the range from about 6 mm Hg to 7 mm Hg.

In an aspect, the present invention provides the aqueous micelle solution according to the preceding embodiment for lowering intraocular pressure in a mammal by at least 4 mm Hg within 2 hours upon instillation into the eye of the mammal.

In an aspect, the present invention provides a method of lowering intraocular pressure in a mammal comprising administering twice daily in the eye of the mammal, the aqueous micelle solution according to the preceding embodiment.

In an aspect, the present invention provides a method of lowering intraocular pressure in a mammal comprising administering once daily in the eye of the mammal, the aqueous micelle solution according to the preceding embodiment.

In an alternative embodiment, the present invention provides an aqueous micelle solution comprising 1.25% w/v Brinzolamide in the form of polymeric micelles, wherein the Brinzolamide is dispersed uniformly in a polymer. The polymer in the micelle is a polyvinyl caprolactam-polyvinyl acetate-polyethylene glycol graft co-polymer. The solution does not contain a surfactant and exhibits higher Tmax and AUC values as compared to conventional standard 1% w/v suspension of Brinzolamide, when the solution and suspension are administered in equal volume, to a mammal by instillation in eye. In a preferred aspect of this embodiment, the Tmax is reached between 3.5 to 4.5 hours after instillation in the eye of the mammal. In another preferred aspect of this embodiment, IOP reduction after instillation of the solution in the eye of the mammal, is in the range from about 6 mm Hg to 7 mm Hg.

In an aspect, the present invention provides the aqueous micelle solution according to the preceding embodiment for lowering intraocular pressure in a mammal by at least 5 mm Hg within 2 hours upon instillation into the eye of the mammal.

In an aspect, the present invention provides a method of lowering intraocular pressure in a mammal comprising administering twice daily in the eye of the mammal, the aqueous micelle solution according to the preceding embodiment.

In an aspect, the present invention provides a method of lowering intraocular pressure in a mammal comprising administering once daily in the eye of the mammal, the aqueous micelle solution according to the preceding embodiment.

In an alternative embodiment, the present invention provides an aqueous micelle solution comprising 0.75% w/v Brinzolamide in the form of polymeric micelles, wherein the Brinzolamide is dispersed uniformly in a polymer. The polymer in the micelle is a polyvinyl caprolactam-polyvinyl acetate-polyethylene glycol graft co-polymer, Wherein the solution does not contain a surfactant and exhibits higher AUC values as compared to conventional standard 1% w/v suspension of Brinzolamide, when the solution and suspension are administered in equal volume, to a mammal by instillation in the eye of the mammal.

In an aspect, the present invention provides a method of lowering intraocular pressure in a mammal comprising administering twice or thrice daily in the eye of the mammal, the aqueous micelle solution according to the preceding embodiment.

In an aspect, the present invention provides the aqueous micelle solution according to the preceding embodiment for lowering intraocular pressure in a mammal by at least 3.5 mm Hg within 2 hours upon instillation into the eye of the mammal.

In an alternative embodiment, the present invention provides an aqueous micelle solution comprising 0.5% w/v Brinzolamide in the form of polymeric micelles; wherein the polymer in the micelle is a polyvinyl caprolactam-polyvinyl acetate-polyethylene glycol graft co-polymer; and wherein the solution does not contain a surfactant.

In an aspect, the present invention provides the aqueous micelle solution according to the preceding embodiment for lowering intraocular pressure in a mammal by at least 3 mm Hg within 2 hours upon instillation into the eye of the mammal.

In an embodiment, the present invention provides a method of preparing an aqueous micelle solution comprising Brinzolamide in the form of polymeric micelles; comprising the step of; homogeneous mixing of a solid dispersion of Brinzolamide and polyvinyl caprolactam-polyvinyl acetate-polyethylene glycol graft co-polymer; and one or more pharmaceutically acceptable excipients; wherein the excipients do not contain a surfactant. In a preferred aspect of this embodiment, the solid dispersion is prepared by solvent evaporation. In another preferred aspect of this embodiment, the solid dispersion is prepared by hot melt extrusion in a twin screw extruder. In another preferred aspect of this embodiment, the weight ratio of Brinzolamide to the polyvinyl caprolactam-polyvinyl acetate-polyethylene glycol graft co-polymer in the solid dispersion is within the range of 1:2.5 to 1:15; preferably 1:10 to 1:15. In another preferred aspect of this embodiment, the aqueous micelle solution is prepared without subjecting it to heat sterilization.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 depicts effect of Brinzolamide compositions (ML1A, ML1B, RLD) on α-Chymotrypsin induced glaucoma in New Zealand Rabbits; and

FIG. 2 depicts effect of Brinzolamide compositions (ML2A and ML2B) on α-Chymotrypsin induced glaucoma in New Zealand Rabbits.

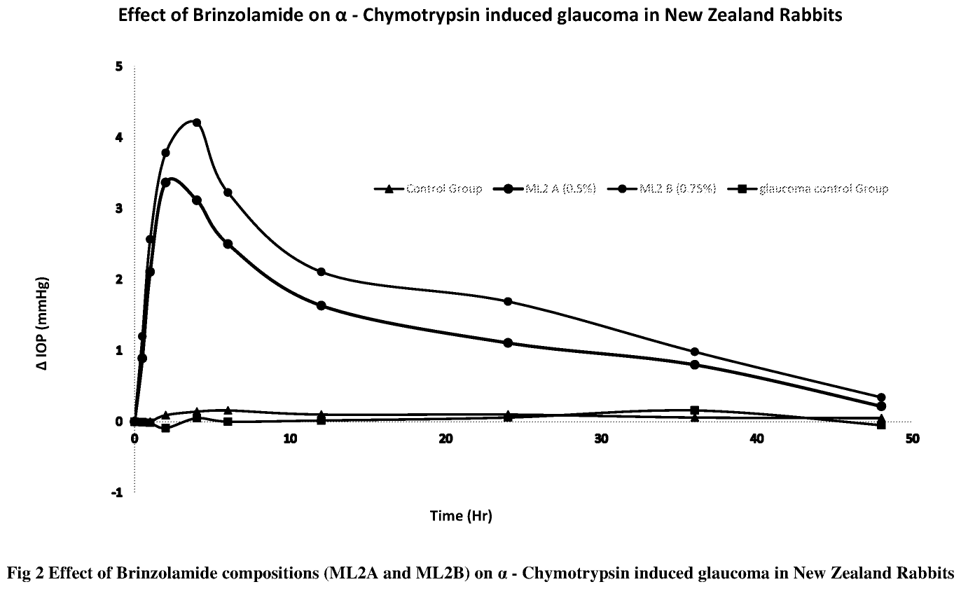

### DETAILED DESCRIPTION OF THE INVENTION

The present invention provides an aqueous micelle solution of Brinzolamide. The term “Brinzolamide” as used in the invention, unless specified otherwise, is meant to cover Brinzolamide in the form of free base or its pharmaceutically acceptable salt(s), hydrate(s), solvate(s) and physiologically functional derivative(s) and precursors thereof.

The present invention provides an ophthalmic solution of Brinzolamide comprising polymeric micelles of Brinzolamide and a polymer.

In an embodiment, the present invention provides an aqueous micelle solution comprising Brinzolamide dispersed uniformly in a polymer; in the form of polymeric micelles; wherein the polymer in the micelle is a polyvinyl caprolactam-polyvinyl acetate-polyethylene glycol graft co-polymer.

Polyvinyl caprolactam-polyvinyl acetate-polyethylene glycol graft copolymer identified by CAS no. 402932-23-4 (available as Soluplus®) is a polymer with amphiphilic properties, suitable for solid solutions. Unlike solubilizers like Cremophor RH40 and Solutol HS15; Soluplus® has a bifunctional character. It is a matrix polymer for solid solutions and is also capable of solubilizing poorly soluble drugs in aqueous media. Soluplus® exhibits a glass transition temperature of about 70° C. The inventors have harnessed these bifunctional characteristics of polyvinyl caprolactam-polyvinyl acetate-polyethylene glycol graft copolymer to initially prepare solid dispersions of Brinzolamide and polyvinyl caprolactam-polyvinyl acetate-polyethylene glycol graft copolymer by homogeneous mixing of Brinzolamide and polyvinyl caprolactam-polyvinyl acetate-polyethylene glycol graft copolymer, and the solid dispersion was used to prepare aqueous solution in which the Brinzolamide with the copolymer is in the form of polymeric micelles.

The homogeneous mixing of Brinzolamide and polyvinyl caprolactam-polyvinyl acetate-polyethylene glycol graft copolymer is done by a solvent evaporation method, or by a hot melt extrusion method.

Soluplus® is a triblock-copolymer, a graft copolymer consisting of polyethylene glycol (PEG), polyvinylcaprolactam, and polyvinyl acetate (13% PEG 6000; 57% vinyl caprolactam; 30% vinyl acetate). It has a PEG 6000 backbone with one or two sidechains consisting of vinyl acetate randomly copolymerized with vinyl caprolactam. It is available as white to yellowish free flowing granules. It has average molecular weight of 118,000 g/mol (nominally in the range of 90,000-140,000 g/mol). It is an amphiphilic copolymer comprising hydrophilic and hydrophobic blocks.

Brinzolamide and polyvinyl caprolactam-polyvinyl acetate-polyethylene glycol graft co-polymer polymeric micelles with core-shell morphology are formed from the copolymer, which are hydrophobic polymer chains linked to hydrophilic polymeric chains. The assembling of the hydrophobic parts of the block copolymer forms the inner micelle core in the aqueous medium through hydrophobic interactions, whereas the outer hydrophilic parts surround the inner core as a hydrated shell.

The polymeric micelles have also contributed to stability of the aqueous Brinzolamide solutions of the present invention. The present invention provides stable aqueous micelle solution comprising Brinzolamide dispersed uniformly in polyvinyl caprolactam-polyvinyl acetate-polyethylene glycol graft co-polymer in the form of polymeric micelles; wherein the solution is stable at harsh conditions of 40° C. and 75% relative humidity for 6 months. The stability is exhibited especially in terms of total impurity levels of not more than 2% by weight of the solution even after being subjected to the harsh conditions mentioned above.

Surprisingly, the Inventors have been able to effectively solubilize Brinzolamide without the use of a surfactant. The Inventors have been also able to effectively solubilize Brinzolamide by using polyvinyl caprolactam-polyvinyl acetate-polyethylene glycol graft co-polymer alone.

In an embodiment, the present invention provides an aqueous micelle solution comprising Brinzolamide dispersed uniformly in a polymer; in the form of polymeric micelles; wherein the polymer in the micelle is a polyvinyl caprolactam-polyvinyl acetate-polyethylene glycol graft co-polymer; and wherein the solution does not contain a surfactant.

In a preferred aspect of any one of the above embodiments, there is provided the ophthalmic solution wherein, the solution further does not contain a solubilizer of the cyclodextrin class of compounds. In a most preferred aspect of any one of the above embodiments, there is provided the ophthalmic solution wherein, the solution further does not contain any solubilizer other than polyvinyl caprolactam-polyvinyl acetate-polyethylene glycol graft co-polymer.

In an embodiment, the present invention provides a method of preparing an aqueous micelle solution comprising Brinzolamide in the form of polymeric micelles; by homogeneous mixing of a solid dispersion of Brinzolamide and polyvinyl caprolactam-polyvinyl acetate-polyethylene glycol graft co-polymer; and one or more pharmaceutically acceptable excipients wherein the pharmaceutically acceptable excipients do not contain a surfactant. In a preferred aspect of this embodiment, the Brinzolamide used in the preparation of the micelle solution has a particle size D₉₀ is in the range of 5 to 10 microns. In a preferred aspect of this embodiment, the polyvinyl caprolactam-polyvinyl acetate-polyethylene glycol graft co-polymer used in the preparation of the micelle solution has average particle size is in the range of 300 to 350 microns. In a preferred aspect of this embodiment, the solid dispersion is prepared by solvent evaporation. In another preferred aspect of this embodiment, the solid dispersion is prepared by hot melt extrusion in a twin screw extruder. In another preferred aspect of this embodiment, the weight ratio of Brinzolamide to the polyvinyl caprolactam-polyvinyl acetate-polyethylene glycol graft co-polymer in the solid dispersion is within the range of 1:2.5 to 1:15; preferably 1:10 to 1:15. In another preferred aspect of this embodiment, the aqueous micelle solution is prepared without subjecting the solution to heat sterilization.

As described herein, the D₁₀ means 10% of the volume of particles have a diameter less than a specified diameter, the D₅₀ means 50% of the volume of particles have a diameter less than a specified diameter and the D₉₀ means 90% of the volume of particles have a diameter less than a specified diameter. Average particle size means average of all particles in the sample equal to or less than a specified diameter. The particle size of the Brinzolamide as well as particle size of the polyvinyl caprolactam-polyvinyl acetate-polyethylene glycol graft co-polymer is measured by laser light scattering technique as known to those skilled in the art.

The one or more pharmaceutically acceptable excipients, are suitably selected from buffering agents, preservatives, tonicity-adjusting agents, pH-adjusting agents, chelating agents, antioxidants and solvents.

Examples of preservatives include, but are not limited to benzalkonium chloride, benzethonium chloride, p-oxybenzoates such as methyl p-oxybenzoate or ethyl p-oxybenzoate, benzyl alcohol, phenethyl alcohol, sorbic acid or its salt, thimerosal, chlorobutanol, other quaternary amines and, chlorhexidine gluconate or a combination thereof.

Examples of tonicity adjusting agents include, but are not limited to polyethylene glycol, propylene glycol, mannitol, glycerine dextrose, trehalose, sucrose, electrolytes selected from sodium chloride, potassium chloride, magnesium chloride or calcium chloride or a combination thereof.

Examples of the alkaline agents that may be used as pH adjusting agents, include, but are not limited to, sodium hydroxide (NaOH), potassium hydroxide (KOH), tromethamine, monoethanolamine, sodium bicarbonate (NaHCO₃) and other organic and inorganic bases or a combination thereof.

Examples of the acidic agents that may be used as pH adjusting agents include, but are not limited to, hydrochloric acid, citric acid, tartaric acid, lactic acid and other organic and inorganic acids or a combination thereof.

Examples of chelating agents include, but are not limited to,

Ethylenediaminetetraacetic acid (EDTA), Disodium Edetate, sodium citrate, condensed sodium phosphate or a combination thereof.

Examples of antioxidants include, but are not limited to, ascorbic acid, malic acid, citric acid, sodium citrate, butylated hydroxyanisole, butylated hydroxytoluene, propyl gallate, sodium ascorbate, sodium metabisulfite or a combination thereof.

Examples of solvents include, but are not limited to, water, ethanol, propylene glycol or a combination thereof.

In a preferred embodiment, a solid dispersion of Brinzolamide is prepared by solvent evaporation. In an alternative embodiment, a solid dispersion of Brinzolamide is prepared by hot melt extrusion through a twin screw extruder.

In yet another embodiment, the present invention provides a process for preparation of the aqueous micelle solution comprising Brinzolamide dispersed uniformly in polyvinyl caprolactam-polyvinyl acetate-polyethylene glycol graft co-polymer, in the form of polymeric micelles, comprising the steps of:
1. mixing of the Brinzolamide and polyvinyl caprolactam-polyvinyl acetate-polyethylene glycol graft co-polymer;
2. adding ethanol gradually in small aliquots to a mixture of the Brinzolamide and the polyvinyl caprolactam-polyvinyl acetate-polyethylene glycol graft co-polymer mixture, till it gets solubilized to form a clear solution;
3. evaporating the ethanol to form a thin film solid dispersion;
4. dispersing the above film in a phosphate buffer saline solution or water to form a solution;
5. adding one or more pharmaceutically acceptable excipients in the solution;
6. passing the obtained solution through a 0.45-micron membrane filter and 0.2-micron membrane filter to make it a sterile ophthalmic solution;
   wherein the pharmaceutically acceptable excipient is not a surfactant.

In an aspect of the preceding embodiment, the evaporation of ethanol is carried out using a rotary evaporator at suitable temperature for suitable time to form a thin film. Preferably, the suitable temperature is 30° C. to 50° C. and time is 3 to 4 hrs.

In yet another embodiment, the present invention provides a process for preparation of the aqueous micelle solution comprising Brinzolamide dispersed uniformly in polyvinyl caprolactam-polyvinyl acetate-polyethylene glycol graft co-polymer, in the form of polymeric micelles, comprising the steps of:
1. mixing of the Brinzolamide and the polyvinyl caprolactam-polyvinyl acetate-polyethylene glycol graft co-polymer to prepare a blend;
2. passing the blend through hot melt extruder at a temperature in the range of 40° C. to 150° C. to prepare a solid dispersion in the form of extrudates;
3. sizing the extrudates through a co-mill;
4. adding the extrudates to water and stirring continuously until completely solubilized;
5. adding one or more pharmaceutically acceptable excipients;
6. passing the obtained solution through a 0.45-micron membrane filter and 0.2-micron membrane filter to make it a sterile ophthalmic solution;
   wherein the pharmaceutically acceptable excipient is not a surfactant.

In an embodiment, the aqueous micelle solutions as per present invention have pH adjusted in the range of 5.5 to 8.0, preferably and its osmolality in the range of 270-350 mOsm/Kg.

Surprisingly, the inventors of the present invention have provided an aqueous micelle 1% w/v Brinzolamide solution comprising polymeric micelles of Brinzolamide; which is more efficacious than conventional standard 1% w/v suspension of Brinzolamide. The efficacy was exhibited by instilling equal volumes (2 drops) of the solution and suspension in the eye of different glaucoma induced test subjects (mammals) and measuring the effect of Brinzolamide, in terms of decrease in Intra-Ocular Pressure Vs time. It was found that the extent of absorption of Brinzolamide from the aqueous micelle 1% w/v Brinzolamide solution, as well as the time required to reach the maximum IOP lowering effect was higher than the conventional standard 1% w/v suspension of Brinzolamide.

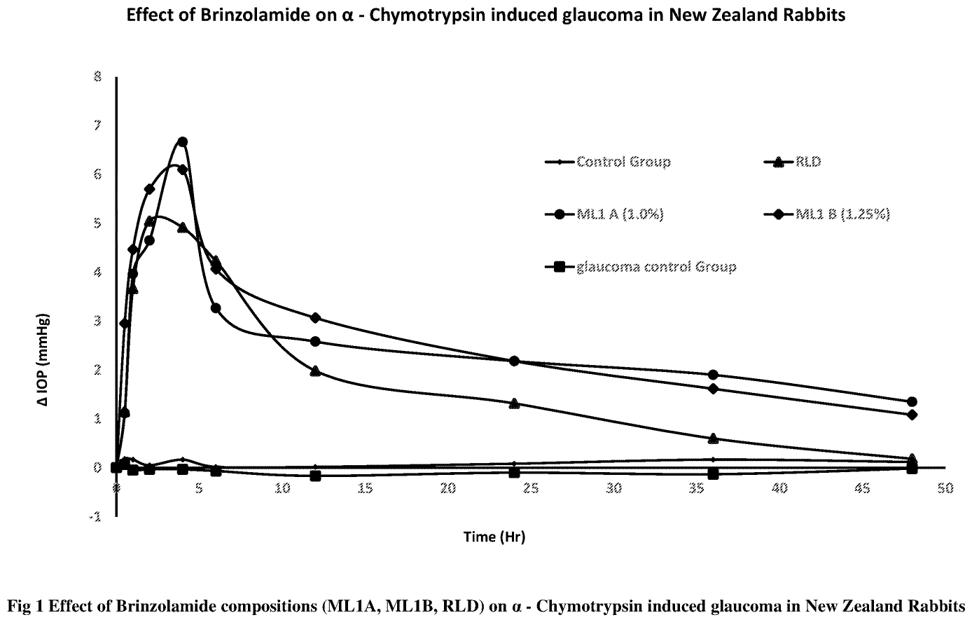

Tmax is the time to reach maximum IOP lowering effect after instillation or in other words Time to reach peak decrease in IOP, and AUC is Area Under the Curve, representing Area Under the ΔIOP vs Time curve. In the present invention the terms Tmax and AUC are to be interpreted in context of and in accordance with FIG. 1, unless otherwise specified.

In an embodiment, the present invention provides an aqueous micelle solution comprising 1% w/v Brinzolamide in the form of polymeric micelles; wherein the Brinzolamide is dispersed uniformly in a polymer; wherein the polymer in the micelle is a polyvinyl caprolactam-polyvinyl acetate-polyethylene glycol graft co-polymer; wherein the solution does not contain a surfactant and exhibits higher Tmax and AUC values as compared to conventional standard 1% w/v suspension of Brinzolamide, when the solution, and the suspension are administered in equal volume, to a mammal by instillation in the eye of the mammal. In a preferred aspect of this embodiment, the Tmax is reached between 3.5 to 4.5 hours after instillation in the eye of the mammal. In another preferred aspect of this embodiment, the AUC is about 117 h mm Hg. In another preferred aspect of this embodiment, IOP reduction in the mammal after instillation of the solution in the eye of the mammal, is in the range from 6 mm Hg to 7 mm Hg. In another preferred aspect of this embodiment, the size of polymeric micelles is such that, D₉₀ is in the range from about 130 nm to 200 nm. In another preferred aspect of this embodiment, the size of polymeric micelles is such that, D₅₀ is in the range from about 70 nm to 100 nm. In another preferred aspect of this embodiment, the size of polymeric micelles is such that, D₁₀ is in the range from about 30 nm to 60 nm. In an aspect, the present invention provides the aqueous micelle solution according to the preceding embodiment, comprising 1% w/v Brinzolamide; wherein the solution lowers intraocular pressure in a mammal by at least 4 mm Hg within 2 hours upon instillation into the eye of the mammal.

The dosage frequency as recommended for conventional standard 1% w/v suspension of Brinzolamide for the treatment of elevated intraocular pressure (IOP) in patients with ocular hypertension or open-angle glaucoma is 2-3 times daily. It can be envisaged that, the aqueous micelle solution comprising 1% w/v Brinzolamide in the form of polymeric micelles, as per preceding embodiment would have to be administered once or twice daily in the patient because it is twice as efficacious as the conventional standard 1% w/v Brinzolamide suspension.

In an aspect, the present invention provides a method of lowering intraocular pressure in a mammal comprising administering once daily in the eye of the mammal, the aqueous micelle solution comprising 1% w/v Brinzolamide in the form of polymeric micelles as per preceding embodiment.

In an aspect, the present invention provides a method of lowering intraocular pressure in a mammal comprising administering twice daily in the eye of the mammal, the aqueous micelle solution comprising 1% w/v Brinzolamide in the form of polymeric micelles as per preceding embodiment.

Similarly, in an alternative embodiment, the present invention provides an aqueous micelle solution comprising 1.25% w/v Brinzolamide in the form of polymeric micelles; wherein the Brinzolamide is dispersed uniformly in a polymer; wherein the polymer in the micelle is a polyvinyl caprolactam-polyvinyl acetate-polyethylene glycol graft co-polymer; wherein the solution does not contain a surfactant and exhibits higher Tmax and AUC values as compared to conventional standard 1% w/v suspension of Brinzolamide, when the solution and suspension are administered in equal volume, to a mammal by instillation in the eye of the mammal. In a preferred aspect of this embodiment, the Tmax is reached between 3.5 to 4.5 hours after instillation in the eye of the mammal. In another preferred aspect of this embodiment, IOP reduction in the mammal after instillation of the solution in the eye, is in the range from 6 mm Hg to 7 mm Hg. In another preferred aspect of this embodiment, the size of polymeric micelles is such that, D₉₀ is in the range from about 130 nm to 200 nm. In another preferred aspect of this embodiment, the size of polymeric micelles is such that, D₅₀ is in the range from about 70 nm to 100 nm. In another preferred aspect of this embodiment, the size of polymeric micelles is such that, D₁₀ is in the range from about 30 nm to 60 nm. In an aspect, the present invention provides the aqueous micelle solution according to the preceding embodiment, comprising 1.25% w/v Brinzolamide; wherein the solution lowers intraocular pressure in a mammal by at least 5 mm Hg within 2 hours upon instillation into the eye of the mammal.

It can be envisaged that, the aqueous micelle solution comprising 1.25% w/v Brinzolamide in the form of polymeric micelles, as per preceding embodiment would have to be administered once or twice daily in the patient because it is twice as efficacious as the conventional standard 1% w/v Brinzolamide suspension.

In an aspect, the present invention provides a method of lowering intraocular pressure in a mammal comprising administering once daily in the eye of the mammal, the aqueous micelle solution comprising 1.25% w/v Brinzolamide in the form of polymeric micelles as per preceding embodiment.

In an aspect, the present invention provides a method of lowering intraocular pressure in a mammal comprising administering twice daily in the eye of the mammal, the aqueous micelle solution comprising 1.25% w/v Brinzolamide in the form of polymeric micelles as per preceding embodiment.

Brinzolamide has a lot of adverse effects reported in the label, such as blepharitis, dermatitis, dry eye, foreign body sensation, headache, hyperemia, ocular discharge, ocular discomfort, ocular keratitis, ocular pain, ocular pruritus, and rhinitis. It would be desirable to decrease the dose of Brinzolamide administered to a patient. Therefore, the inventors have provided lower strengths of Brinzolamide solution viz., aqueous micelle solutions of strengths 0.5% w/v Brinzolamide and 0.75% w/v Brinzolamide.

In an alternative embodiment, the present invention provides an aqueous micelle solution comprising 0.5% w/v or 0.75% w/v Brinzolamide in the form of polymeric micelles; wherein the polymer in the micelle is a polyvinyl caprolactam-polyvinyl acetate-polyethylene glycol graft co-polymer; and wherein the solution does not contain a surfactant. In a preferred aspect of this embodiment, the size of polymeric micelles is such that, D₉₀ is in the range from about 130 nm to 200 nm. In another preferred aspect of this embodiment, the size of polymeric micelles is such that, D₅₀ is in the range from about 70 nm to 100 nm. In another preferred aspect of this embodiment, the size of polymeric micelles is such that, D₁₀ is in the range from about 30 nm to 60 nm.

In an aspect, the present invention provides a method of lowering intraocular pressure in a mammal comprising administering thrice daily in the eye of the mammal, the aqueous micelle solution according to the preceding embodiment.

Surprisingly, the inventors have provided aqueous micelle solution of strength 0.75% w/v Brinzolamide which have similar efficacy as the conventional standard 1% w/v Brinzolamide suspension.

In an alternative embodiment, the present invention provides an aqueous micelle solution comprising 0.75% w/v Brinzolamide in the form of polymeric micelles; wherein the Brinzolamide is dispersed uniformly in a polymer; wherein the polymer in the micelle is a polyvinyl caprolactam-polyvinyl acetate-polyethylene glycol graft co-polymer; wherein the solution does not contain a surfactant and exhibits higher AUC value as compared to conventional standard 1% w/v suspension of Brinzolamide, when the solution and suspension are administered in equal volume, to a mammal by instillation in eye. In a preferred aspect of this embodiment, the size of polymeric micelles is such that, D₉₀ is in the range from about 130 nm to 200 nm. In another preferred aspect of this embodiment, the size of polymeric micelles is such that, D₅₀ is in the range from about 70 nm to 100 nm. In another preferred aspect of this embodiment, the size of polymeric micelles is such that, D₁₀ is in the range from about 30 nm to 60 nm. In an aspect, the present invention provides the aqueous micelle solution according to the preceding embodiment, comprising 0.75% w/v Brinzolamide; wherein the solution lowers intraocular pressure in a mammal by at least 3.5 mm Hg within 2 hours upon instillation into the eye of the mammal.

As the efficacy of the aqueous micelle solution comprising 0.75% w/v Brinzolamide is the similar to the conventional standard 1% w/v suspension of Brinzolamide. It can be envisaged that the aqueous micelle solution comprising 0.75% w/v Brinzolamide can be a suitable treatment option to replace conventional standard 1% w/v suspension of Brinzolamide.

In an aspect, the present invention provides a method of lowering intraocular pressure in a mammal comprising administering thrice daily in the eye of the mammal, the aqueous micelle solution according to the preceding embodiment.

In an alternative embodiment, the present invention provides an aqueous micelle solution comprising 0.5% w/v Brinzolamide in the form of polymeric micelles; wherein the Brinzolamide is dispersed uniformly in a polymer; wherein the polymer in the micelle is a polyvinyl caprolactam-polyvinyl acetate-polyethylene glycol graft co-polymer; wherein the solution does not contain a surfactant. In another preferred aspect of this embodiment, the size of polymeric micelles is such that, D₉₀ is in the range from about 130 nm to 200 nm. In another preferred aspect of this embodiment, the size of polymeric micelles is such that, D₅₀ is in the range from about 70 nm to 100 nm. In another preferred aspect of this embodiment, the size of polymeric micelles is such that, D₁₀ is in the range from about 30 nm to 60 nm. In an aspect, the present invention provides the aqueous micelle solution according to the preceding embodiment, comprising 0.5% w/v Brinzolamide; wherein the solution lowers intraocular pressure in a mammal by at least 3 mm Hg within 2 hours upon instillation into the eye of the mammal.

In an aspect, the present invention provides a method of lowering intraocular pressure in a mammal comprising administering at least thrice daily in the eye of the mammal, the aqueous micelle solution according to the preceding embodiment.

Advantageously, the ophthalmic solution obtained through the present invention are milky white or light bluish aqueous solutions because of the complete dissolution of Brinzolamide in the polymeric micelle systems.

In an embodiment, the polymeric micelle size distribution was specified to be maintained in a narrow range of about 30 nm to 200 nm.

In an aspect of the preceding embodiment, the polymeric micelles are in nano-sized and have the following size distribution; D₁₀ is in the range from about 30 nm to 60 nm, D₅₀ is in the range from about 70 nm to 100 nm, and D₉₀ is in the range from about 130 nm to 200 nm.

In another aspect of the preceding embodiment, the polymeric micelles have a following size distribution; wherein D₁₀ is in the range of 30-50 nm, D₅₀ is in the range of 55 to 75 nm and D₉₀ is in the range of 75-140 nm.

It is preferable that, the size distribution of the polymeric micelles in the aqueous micelle solutions of Brinzolamide 0.5, 0.75, 1 and 1.75% w/v disclosed as per present invention; is such that, D₉₀ is in the range from about 100-170 nm.

The size distribution of polymeric micelles is measured by Dynamic light scattering (DLS) method as known to those skilled in the art.

The aqueous micelle solutions as per present invention can be packaged in single use or single dose containers and multi-use or multi dose container. For the aqueous micelle solutions containing preservatives, the containers could be selected from those made from polyethylene material, polypropylene material and polypropylene-polyethylene copolymer material. Preferably, white ethylene oxide sterilized LDPE bottles are used to package 10 ml volume of the solutions disclosed as per present invention.

In an embodiment, the present invention relates to an aqueous micelle solution comprising Brinzolamide dispersed uniformly in a polymer; in the form of polymeric micelles; wherein the polymer in the micelle is a polyvinyl caprolactam-polyvinyl acetate-polyethylene glycol graft co-polymer; wherein the solution does not contain a surfactant, and does not contain any preservative.

By selecting a suitable packaging, preservative free solutions as per the preceding embodiment can be packaged in sterile single use preservative free containers or sterile multi-dose preservative free containers; such as multi-dose containers with nozzle design such that it has a non-return valve, a non-return piston or a membrane filter; and/or actuation that is variable or discreet are suitable for packaging.

There are known impurities associated with Brinzolamide viz., Impurity A, B, G and H.
i. Impurity A-(4S)-4-(Ethylamino)-2-(3-methoxypropyl)-3,4-dihydro-2H-thieno[3,2-e][1,2]thiazine-6-sulfonamide 1,1-dioxide

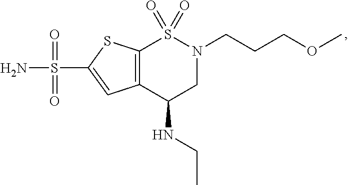 [SMILES: CCN[C@@H]1CN(CCCOC)S(=O)(=O)c2sc(S(N)(=O)=O)cc21]

ii. Impurity B-(4R)-4-Amino-2-(3-methoxypropyl)-3,4-dihydro-2H-thieno[3,2-e][1,2]thiazine-6-sulfonamide 1,1-dioxide ethanedioate

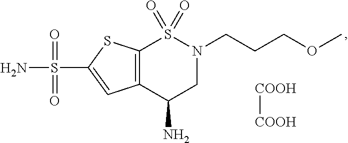

iii. Impurity G-(4R)-2-(3-Methoxypropyl)-4-(methylamino)-3,4-dihydro-2H-thieno[3,2-e][1,2]thiazine-6-sulfonamide 1,1-dioxide

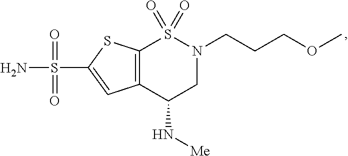 [SMILES: CN[C@H]1CN(CCCOC)S(=O)(=O)c2sc(S(N)(=O)=O)cc21]

and iv.
Impurity H-(R)-4-[Ethyl(methylsulfonyl)amino]-2-(3-methoxypropyl)-3,4-dihydro-2H-thieno[3,2-e]1,2-thiazine-6-sulfonamide 1,1-dioxide

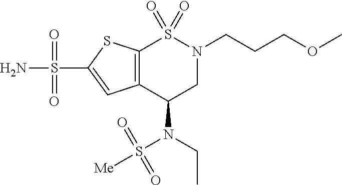 [SMILES: CCN([C@@H]1CN(CCCOC)S(=O)(=O)c2sc(S(N)(=O)=O)cc21)S(C)(=O)=O]

In an embodiment, the present invention relates to an aqueous micelle solution comprising Brinzolamide dispersed uniformly in a polymer; in the form of polymeric micelles; wherein the polymer in the micelle is a polyvinyl caprolactam-polyvinyl acetate-polyethylene glycol graft co-polymer; wherein the solution does not contain a surfactant; wherein the amount of total impurities in the solution, does not exceed 2% w/v after storage for 3 months in a closed container; at 40° C. and 75% RH (Relative Humidity); or 30° C. and 65% RH; or 25° C. and 40% RH.

In an embodiment, the present invention relates to an aqueous micelle solution comprising Brinzolamide dispersed uniformly in a polymer; in the form of polymeric micelles; wherein the polymer in the micelle is a polyvinyl caprolactam-polyvinyl acetate-polyethylene glycol graft co-polymer; wherein the solution does not contain a surfactant; wherein the amount of Impurity A in the solution, does not exceed 1% w/v after storage for 3 months in a closed container; at 40° C. and 75% RH (Relative Humidity); or 30° C. and 65% RH; or 25° C. and 40% RH.

In an embodiment, the present invention relates to an aqueous micelle solution comprising Brinzolamide dispersed uniformly in a polymer; in the form of polymeric micelles; wherein the polymer in the micelle is a polyvinyl caprolactam-polyvinyl acetate-polyethylene glycol graft co-polymer; wherein the solution does not contain a surfactant; wherein the amount of Impurity B in the solution, does not exceed 0.15% w/v after storage for 3 months in a closed container; at 40° C. and 75% RH (Relative Humidity); or 30° C. and 65% RH; or 25° C. and 40% RH.

In an embodiment, the present invention relates to an aqueous micelle solution comprising Brinzolamide dispersed uniformly in a polymer; in the form of polymeric micelles; wherein the polymer in the micelle is a polyvinyl caprolactam-polyvinyl acetate-polyethylene glycol graft co-polymer; wherein the solution does not contain a surfactant; wherein the amount of Impurity G in the solution, does not exceed 0.1% w/v after storage for 3 months in a closed container; at 40° C. and 75% RH (Relative Humidity); or 30° C. and 65% RH; or 25° C. and 40% RH.

In an embodiment, the present invention relates to an aqueous micelle solution comprising Brinzolamide dispersed uniformly in a polymer; in the form of polymeric micelles; wherein the polymer in the micelle is a polyvinyl caprolactam-polyvinyl acetate-polyethylene glycol graft co-polymer; wherein the solution does not contain a surfactant; wherein the amount of Impurity H in the solution, does not exceed 0.1% w/v after storage for 3 months in a closed container; at 40° C. and 75% RH (Relative Humidity); or 30° C. and 65% RH; or 25° C. and 40% RH.

In can be understood to a person skilled in the art from the disclosed embodiments of the present invention, that suitable alternatives to polyvinyl caprolactam-polyvinyl acetate-polyethylene glycol graft co-polymer could be used to prepare polymeric micelles of Brinzolamide without deviating from the teachings of the present invention. Polyvinyl caprolactam-polyvinyl acetate-polyethylene glycol graft co-polymer can be replaced by suitable polymers selected from any one of polyoxyl lipid or fatty acid (polyoxyl castor oil-HCO-40, HCO-60, HCO-80 or HCO-100), polyethylene glycol monostearate (Gelucire® 48/16), copovidone (Kollidon VA 64), povidone (Kollidon 12 PF, Kollidon 17 PF, Kollidon 30 and Kollidon 90 F), polyvinyl acetate-povidone (Kollidon SR), polyethylene glycol-polyvinyl alcohol grafted copolymer (Kollicoat IR), methacrylate polymers (Eudragit EPO), polyethylene oxide, polyvinyl acetate, hydroxypropyl methyl cellulose acetate succinate, hydroxypropyl methylcellulose, hydroxypropyl cellulose.

The different embodiments of the present invention are further illustrated by following non-limiting examples.

### Example 1

### Preparation of Ophthalmic Solution by Thin Film Hydration Method Using Rotary Evaporator

Brinzolamide to polyvinyl caprolactam-polyvinyl acetate-polyethylene glycol graft co-polymer ratios for preparation of ophthalmic solution composition are mentioned in the table 1. The method used was thin film hydration method using composition given in table 1.

TABLE 1
Composition of ophthalmic solution for Brinzolamide using
polyvinyl caprolactam-polyvinyl acetate-polyethylene glycol
graft co-polymer by thin film hydration approach

| | | Quantity | | | | | | | |
|---|---|---|---|---|---|---|---|---|---|
| Sr. | | Example 1A | | Example 1B | | Example 1C | | Example 1D | |
| No. | Ingredients | (1:2.5) | | (1:5) | | (1:10) | | (1:15) | |
| 1. | Brinzolamide | 500 | mg | 500 | mg | 500 | mg | 500 | mg |
| 2. | Soluplus ®* | 1250 | mg | 2500 | mg | 5000 | mg | 7500 | mg |
| 3. | Ethanol^{#} | 25 | ml | 30 | ml | 70 | ml | 70 | ml |
| 4. | Phosphate Buffer | 50 | ml | 50 | ml | 50 | ml | 50 | ml |

Solution, pH 7.4

*Soluplus ® is the polyvinyl caprolactam-polyvinyl acetate-polyethylene glycol graft co-polymer
^{#}Ethanol was evaporated during procedure.

### Manufacturing Procedure:

1. Brinzolamide and Soluplus® were dispensed in a quantity as mentioned in Table 1.
2. Soluplus® was added gradually in required amount of ethanol as mentioned in Table 1 under stirring. The stirring was continued till it gets completely solubilized.
3. Brinzolamide was added to the solution of step 2 and stirred until clear solution was formed.
4. The solution obtained in the step 3 was transferred to round bottom flask of rotary evaporator and ethanol was evaporated under low pressure, in a rotary evaporator set at a temperature of 50° C. for 3-4 hrs, to get a thin film.
5. The obtained thin film was rehydrated with phosphate buffer solution, pH 7.4 to form polymeric micelles, using rotary evaporator at 250 RPM until complete solubilisation of thin film and homogenous solution obtained.
6. The volume was made up to 50 ml using phosphate buffer solution, pH 7.4.
7. This solution was then filtered through 0.45-micron membrane filter to obtain final composition.

The physicochemical properties of ophthalmic solution of Brinzolamide as prepared in example 1 are given below in Table 2.

TABLE 2
Physicochemical properties of ophthalmic solution of Brinzolamide
using Soluplus ® by thin film hydration approach

| | | Observation/Results | | | | |
|---|---|---|---|---|---|---|
| Sr. | | Example 1A | Example 1B | Example 1C | | Example 1D |
| No. | Stage | (1:2.5) | (1:5) | (1:10) | | (1:15) |
| 1. | Thin Film appearance | Transparent | Transparent | Transparent | | Transparent |
| | | pale yellow | pale yellow | pale yellow | | pale yellow |
| | | coloured | coloured | coloured | | coloured |
| 2. | Thin film texture | Rubbery | Rubbery | No rubbery | | No rubbery |
| | during rehydration | nature with | nature with | nature, easily | | nature, easily |
| | | difficulty in | difficulty in | solubilized | | solubilized |
| | | solublization | solublization | | | |
| 3. | Appearance of | Milky white | Milky white | Milky white | | Milky white |
| | polymeric micellar | aqueous | aqueous | aqueous | | aqueous |
| | solution | solution | solution | solution | | solution |
| 4. | Assay | | | 93.20% | | |
| 5. | Zeta potential | | | −9.38 | mV | |
| 6. | Size of the polymeric | | | | | |
| | micelles | | | | | |
| | D (10) | | | 46.0 | nm | |
| | D (50) | | | 75.0 | nm | |
| | D (90) | | | 136.0 | nm | |
| | Z average | | | 73.65 | nm | |

### Example 2

Preparation of Brinzolamide solid dispersion by Hot melt extrusion (HME) method. Brinzolamide and Soluplus® were mixed and processed through a hot melt extruder for preparation of solid dispersion. The composition is given in Table 3.

TABLE 3
Composition of solid dispersion of Brinzolamide using
polyvinyl caprolactam-polyvinyl acetate-polyethylene
glycol graft co-polymer by Hot melt extrusion method

| Sr. No. | Ingredients | Quantity (gm) |
|---|---|---|
| 1. | Brinzolamide | 7.00 |
| 2. | Soluplus ®* | 70.00 |

*Soluplus ® is the polyvinyl caprolactam-polyvinyl acetate-polyethylene glycol graft co-polymer in this example.

### Manufacturing Procedure:

1. Brinzolamide and Soluplus® were dispensed as per the aforementioned formula in Table 3 and were mixed uniformly.
2. Physical mixture from step 1 was processed through a hot melt extruder (Thermo Fisher scientific Model: Pharma 11) at different temperature zones ranging from 40° C. to 150° C. The processing parameters along with temperature zones are given in Table 4. An increasing temperature gradient from low to high was maintained from inlet to outlet.
3. The extrudates obtained in the step 2 were dried followed by size reduction using co-mill.
4. The powder obtained in the step 3 was analyzed for assay and loss on drying.

The physicochemical properties of Solid dispersion of Brinzolamide using Soluplus® as prepared in example 2 are given below in Table 5

TABLE 4
Temperatures zones for HME process

| Zone | Temperature | |
|---|---|---|
| 2 | 40° | C. |
| 3 | 70° | C. |
| 4 | 110° | C. |
| 5 | 140° | C. |
| 6 | 150° | C. |
| 7 | 150° | C. |
| 8 | 150° | C. |
| Die | 150° | C. |
| Feed rate | 11 | RPM |
| Extruder screw speed | 100 | RPM |
| Run Time | 20 | min |

TABLE 5
Physicochemical properties of Solid dispersion of
Brinzolamide using Soluplus ® by HME

| Sr. | | |
|---|---|---|
| No. | Stage | Observation/Results |
| 1. | Physical mixture | White coloured powder mixture |
| 2. | Extrudates | Pale yellow or yellow coloured |
| | | glassy extrudes |
| 3. | Assay (%) | 88.90 |
| 4. | Drug Loading (%) | 8.08 |
| 5. | Loss on drying (%) | 0.28 |

### Example 3

### Preparation of Polymeric Micelles Using HME Extrudates.

Solid dispersion prepared using HME Method (Example 2) was used for the preparation of micellar solution. The composition is mentioned in Table 6.

TABLE 6
Composition of Ophthalmic solution of Brinzolamide comprising
solid dispersion prepared by HME prepared in Example 3.

| Sr. No. | Ingredients | Quantity (mg/mL) |
|---|---|---|
| 1. | Brinzolamide solid dispersion | 124.00* |
| 2. | Water for Injection | Q.S. to 1 mL |

*Quantity calculated as per assay obtained in results of Example 2
Q.S—Quantity Sufficient

### Manufacturing Procedure:

1. The water for injection in the quantity of 0.75 ml was dispensed in a vessel.
2. The calculated quantity of Brinzolamide solid dispersion as mentioned in Table 6 was dispensed.
3. The content of step 2 was added to step 1 under continuous stirring until clear solution.
4. The final volume was made to 1 ml using Water for Injection.
5. This solution was then filtered through 0.45-micron membrane filter to obtain final composition.

The physicochemical properties of ophthalmic solution of Brinzolamide as prepared in example 3 are given below in table 7.

TABLE 7
Physicochemical properties of of ophthalmic solution
of Brinzolamide comprising solid dispersion
prepared by HME prepared in Example 3.

| Sr. | | |
|---|---|---|
| No. | Stage/test | Observation |
| 1. | Appearance of solution | Translucent with a |
| | | light bluish tinge |
| 2. | Assay (%) | 93.00 |
| 3. | Size of the polymeric | |
| | micelles | |
| | D (10) | 40.5 nm |
| | D (50) | 56.5 nm |
| | D (90) | 78.2 nm |
| | Z average | 55.36 nm |
| 4. | Zeta potential (mV) | −21.3 |
| 5. | Polydispersity index | 0.018 |

### Example 4

TABLE 8
Composition of solid dispersion of Brinzolamide using
polyvinyl caprolactam-polyvinyl acetate-polyethylene
glycol graft co-polymer by Hot melt extrusion (HME)
method Composition of solid dispersion

| Sr. No. | Ingredients | Quantity (gm) |
|---|---|---|
| 1. | Brinzolamide | 7.00 |
| 2. | Soluplus ®* | 70.00 |

*Soluplus ® is the polyvinyl caprolactam-polyvinyl acetate-polyethylene glycol graft co-polymer in this example.

### Manufacturing Procedure:

5. Brinzolamide and Soluplus® were dispensed as per the aforementioned formula in Table 8 and were mixed uniformly.
6. Physical mixture from step 1 was processed through a hot melt extruder (Thermo Fisher scientific Model: Pharma 11) at different temperature zones ranging from 40° C. to 150° C. The processing parameters along with temperature zones are given in Table 9. An increasing temperature gradient from low to high was maintained from inlet to outlet.
7. The extrudates obtained in the step 2 were dried followed by size reduction using co-mill (Sieve no. 62 G at 3000 RPM)
8. The powder obtained in the step 3 was analyzed for assay and loss on drying. The physicochemical properties of Solid dispersion of Brinzolamide using Soluplus® as prepared in example 4 are given below in Table 5

TABLE 9
Temperatures zones for HME process

| Zone | Temperature | |
|---|---|---|
| 2 | 40° | C. |
| 3 | 70° | C. |
| 4 | 110° | C. |
| 5 | 140° | C. |
| 6 | 150° | C. |
| 7 | 150° | C. |
| 8 | 150° | C. |
| Die | 150° | C. |
| Feed rate | 11 | RPM |
| Extruder screw speed | 100 | RPM |
| Run Time | 20 | min |

TABLE 10
Physicochemical properties of Solid dispersion of
Brinzolamide using Soluplus ® by HME

| Sr. No. | Stage | Observation/Results |
|---|---|---|
| 1. | Physical mixture | White coloured powder mixture |
| 2. | Extrudates | Pale yellow or yellow coloured |
| | | glassy extrudes |
| 3. | Assay (%) | 94.8 |
| 4. | Drug Loading (%) | 8.78 |
| 5. | Loss on drying (%) | 0.93 |

### Example 5

### Preparation of Brinzolamide Ophthalmic Solution, 0.5% w/v (ML2A)

TABLE 11
Quantitative composition for Brinzolamide
Ophthalmic Solution, 0.5% w/v

| Sr. No. | Ingredients | Quantity (mg/mL) |
|---|---|---|
| 1. | Brinzolamide solid dispersion* | 58.56 |
| | (Equivalent to 5 mg/mL Brinzolamide) | |
| 2. | Sodium Chloride | 8 |
| 3. | Benzalkonium Chloride | 0.1 |
| 4. | Water for injection | q.s. to 1 mL |

*Solid dispersion of Example 4: Extrudes of Brinzolamide and Soluplus ® in the ratio of 1:10

The ingredients were weighed according to the batch size as per quantitative composition in Table 11
1. Weighed quantity of Brinzolamide solid dispersion was dissolved in 70% water for injection under continuous stirring to completely dissolve the solid dispersion.
2. Weighed quantity of sodium chloride was added to the solution of Brinzolamide solid dispersion, and stirred until clear solution was obtained.
3. Step 2 was followed by adding, weighed quantity of benzalkonium chloride and stirring until clear solution was obtained.
4. Volume was made up with water for injection to 95% of batch size and stirred.
5. pH of the solution was measured and adjusted to 6.5-7.5 with 1 N NaOH or 0.1 N HCl
6. Volume was made up to batch size with water for injection
7. The solution was filtered through 0.2-micron PES (Polyethersulfone) membrane filter and filled in 10 mL white ETO-sterile LDPE bottles.

### Example 6

### Preparation of Brinzolamide Ophthalmic Solution, 0.75% w/v (ML2B)

TABLE 12
Quantitative composition for Brinzolamide
Ophthalmic Solution, 0.75% w/v

| Sr. No. | Ingredients | Quantity (mg/mL) |
|---|---|---|
| 1. | Brinzolamide solid dispersion* | 87.85 |
| | (Equivalent to 7.5 mg/mL Brinzolamide) | |
| 2. | Sodium Chloride | 7.5 |
| 3. | Benzalkonium Chloride | 0.1 |
| 4. | Water for injection | q.s. to 1 mL |

*Solid dispersion of Example 4: Extrudes of Brinzolamide and Soluplus ® in the ratio of 1:10

The ingredients were weighed according to the batch size as per quantitative composition in Table 12
1. Weighed quantity of Brinzolamide solid dispersion was dissolved in 70% water for injection under continuous stirring to completely dissolve the solid dispersion.
2. Weighed quantity of sodium chloride was added to the solution of Brinzolamide solid dispersion, and stirred until clear solution was obtained.
3. Step 2 was followed by adding, weighed quantity of benzalkonium chloride and stirring until clear solution was obtained.
4. Volume was made up with water for injection to 95% of batch size and stirred.
5. pH of the solution was measured and adjusted to 6.5-7.5 with 1 N NaOH or 0.1 N HCl
6. Volume was made up to batch size with water for injection
7. The solution was filtered through 0.2-micron PES (Polyethersulfone) membrane filter and filled in 10 mL white ETO-sterile LDPE bottles.

### Example 7

TABLE 13
Composition of solid dispersion of Brinzolamide using
polyvinyl caprolactam-polyvinyl acetate-polyethylene
glycol graft co-polymer by Hot melt extrusion (HME)
method Composition of solid dispersion

| Sr. No. | Ingredients | Quantity (gm) |
|---|---|---|
| 1. | Brinzolamide | 7.00 |
| 2. | Soluplus ®* | 70.00 |

*Soluplus ® is the polyvinyl caprolactam-polyvinyl acetate-polyethylene glycol graft copolymer in this example.

### Manufacturing Procedure:

1. Brinzolamide and Soluplus® were dispensed as per the aforementioned formula in Table 8 and were mixed uniformly.
2. Physical mixture from step 1 was processed through a hot melt extruder (Thermo Fisher scientific Model: Pharma 11) at different temperature zones ranging from 40° C. to 150° C. The processing parameters along with temperature zones are given in Table 9. An increasing temperature gradient from low to high was maintained from inlet to outlet.
3. The extrudates obtained in the step 2 were dried followed by size reduction using co-mill (Sieve no. 62 G at 3000 RPM)
4. The powder obtained in the step 3 was analyzed for assay and loss on drying.

The physicochemical properties of Solid dispersion of Brinzolamide using Soluplus® as prepared in example 7 are given below in Table 15

TABLE 14
Temperatures zones for HME process

| Zone | Temperature | |
|---|---|---|
| 2 | 40° | C. |
| 3 | 70° | C. |
| 4 | 110° | C. |
| 5 | 140° | C. |
| 6 | 150° | C. |
| 7 | 150° | C. |
| 8 | 150° | C. |
| Die | 150° | C. |
| Feed rate | 11 | RPM |
| Extruder screw speed | 100 | RPM |
| Run Time | 20 | min |

TABLE 15
Physicochemical properties of Solid dispersion of
Brinzolamide using Soluplus ® by HME

| Sr. No. | Stage | Observation/Results |
|---|---|---|
| 1. | Physical mixture | White coloured powder mixture |
| 2. | Extrudates | Pale yellow or yellow coloured |
| | | glassy extrudes |
| 3. | Assay (%) | 88.9 |
| 4. | Drug Loading (%) | 8.08 |
| 5. | Loss on drying (%) | 0.28 |

### Example 8

### Preparation of Brinzolamide Ophthalmic Solution, 1.0% w/v (ML1A)

TABLE 16
Quantitative composition for Brinzolamide
Ophthalmic Solution, 1.0% w/v

| Sr. No. | Ingredients | Quantity (mg/mL) |
|---|---|---|
| 1. | Brinzolamide solid dispersion* | 116.67 |
| | (Equivalent to 10 mg/mL Brinzolamide) | |
| 2. | Sodium Chloride | 7 |
| 3. | Benzalkonium Chloride | 0.1 |
| 4. | Water for injection | q.s. to 1 mL |

*Solid dispersion of Example 7: Extrudes of Brinzolamide and Soluplus ® in the ratio of 1:10

The ingredients were weighed according to the batch size as per quantitative composition in Table 16
1. Weighed quantity of Brinzolamide solid dispersion was dissolved in 70% water for injection under continuous stirring to completely dissolve the solid dispersion.
2. Weighed quantity of sodium chloride was added to the solution of Brinzolamide solid dispersion, and stirred until clear solution was obtained.
3. Step 2 was followed by adding, weighed quantity of benzalkonium chloride and stirring until clear solution was obtained.
4. Volume was made up with water for injection to 95% of batch size and stirred.
5. pH of the solution was measured and adjusted to 6.5-7.5 with 1N NaOH or 0.1 N HCl
6. Volume was made up to batch size with water for injection
7. The solution was filtered through 0.2-micron PES (Polyethersulfone) membrane filter and filled in 10 mL white ETO-sterile LDPE bottles.

### Cryo-Transmission Electron Microscopy (Cryo-TEM) Study:

Polymeric micelles in the solution of Example 6, were characterized by Cryo-Transmission Electron Microscopy (Cryo-TEM)

The method is based on the rapid freezing of samples at temperatures of at least −170° C. using liquid nitrogen, liquid propane or liquid helium.
Equipment: Talos™ Arctica™ 200 kV TEM Microscope
As per Cryo-TEM results, the micelle size of sample was found to be 30-40 nm.

### Small Angle X-Ray Scattering (SAXS) Study:

Small Angle X-ray Scattering (SAXS) was performed on sample of Example 6, to investigate the structure of polymeric micelles in solution, to observe their size, shape and inner architecture on the 1-100 nm length-scale; and to calculate the aggregation number i.e. the average number of polymers in each micelle

TABLE 17
Observations

| Model | Spherical | |
|---|---|---|
| Sub-model | Core- multi Shell | |
| Total Radius (Å) | 274.1 | (27.41 nm) |
| Radius of core (Å) | 102.7 | (10.27 nm) |
| Number of shells | 4 | |
| Average size of shell (Å) | 42.8 | (4.28 nM) |

SAXS study: Performed on Xenocs SAS,
Model: Xeuss 2.0
Results: Spherical morphology was confirmed by SAXS with Spherical core multi shell model with size 42.8° A

### Example 9

### Preparation of Brinzolamide Ophthalmic Solution, 1.25% w/v (ML1B)

TABLE 18
Quantitative composition for Brinzolamide
Ophthalmic Solution, 1.25% w/v

| Sr. No. | Ingredients | Quantity (mg/mL) |
|---|---|---|
| 1. | Brinzolamide solid dispersion* | 145.84 |
| | (Equivalent to 12.5 mg/mL Brinzolamide) | |
| 2. | Sodium Chloride | 7 |
| 3. | Benzalkonium Chloride | 0.1 |
| 4. | Water for injection | QS to 1 mL |

*Solid dispersion of Example 7: Extrudes of Brinzolamide and Soluplus ® in the ratio of 1:10

The ingredients were weighed according to the batch size as per quantitative composition in Table 18
1. Weighed quantity of Brinzolamide solid dispersion was dissolved in 70% water for injection under continuous stirring to completely dissolve the solid dispersion.
2. Weighed quantity of sodium chloride was added to the solution of Brinzolamide solid dispersion, and stirred until clear solution was obtained.
3. Step 2 was followed by adding, weighed quantity of benzalkonium chloride and stirring until clear solution was obtained.
4. Volume was made up with water for injection to 95% of batch size and stirred.
5. pH of the solution was measured and adjusted to 6.5-7.5 with 1 N NaOH or 0.1 N HCl
6. Volume was made up to batch size with water for injection
7. The solution was filtered through 0.2-micron PES (Polyethersulfone) membrane filter and filled in 10 mL white ETO-sterile LDPE bottles.

### Example 10

### Effect of Brinzolamide Formulations on Ocular Irritation Test in Rabbits

The objective of the study was to evaluate the Acute Ocular Irritation of ML1 A (Brinzolamide 1.0%) and ML1B (Brinzolamide 1.25%) in New Zealand Rabbit

### Ocular Irritation Test in Rabbits

As per OECD guideline 405
Animals: New Zealand White Rabbits (Weight: 1-3 Kg) were divided in different groups as given below
A) Two Test groups were administered test solutions:
   ML1A (Brinzolamide Ophthalmic Solution, 1.0% w/v)
   ML1B (Brinzolamide Ophthalmic Solution, 1.25% w/v)
B) One Control group: was administered a topical anesthetic but did not receive the test solution.
For Initial test: One animal in each group
For Confirmatory test: Two animals in each group
For Confirmatory test, procedure as below was followed for the Test and Control groups.
   The observations were made at 1 h, 24 h, 48 h and 72 h for ocular lesions of cornea, iris, conjunctivae and chemosis.
   One hour before the test solution application, 0.01 mg/kg buprenorphine hydrochloride (0.3 mg/ml), a systemic analgesic was administered by subcutaneous route to both the animals of each group.
   Five min prior to the test solution application, two drops of Proparacaine HCl (0.5%), a topical anesthetic were instilled to both the eyes of each rabbit.
   Two drops of ML1A (Brinzolamide Ophthalmic Solution, 1.0% w/v) and ML1B (Brinzolamide Ophthalmic Solution, 1.25% w/v) were instilled to the respective rabbits, into the conjunctival sac by gently pulling the lower eyelid and then the lids were gently held together for 10 seconds to prevent the loss of test substance.
   Eight hours after test chemical application, 0.01 mg/kg buprenorphine HCl (0.3 mg/ml) and 0.5 mg/kg Meloxicam (0.05 mg/ml) were administered by subcutaneous route to each rabbit.
      The observations were made for three consecutive days at specific time intervals for ocular lesions and scoring was given according to OECD guideline for the testing of chemicals, OECD TG 405.

TABLE 19
Grading for Acute Ocular Irritation of ML1
A and ML1 B as per OECD TG 405 Initial test

| | |
|---|---|
| Time | Gradings |

| Points | Parameters | Observations | ML1A | ML1B |
|---|---|---|---|---|
| 1 h | Cornea | No ulceration | 0 | 0 |
| | | or opacity | | |
| | Iris | Normal | 0 | 0 |
| | Conjunctivae | Normal | 0 | 0 |
| | Chemosis | Normal | 0 | 0 |
| 24 h | Cornea | No ulceration | 0 | 0 |
| | | or opacity | | |
| | Iris | Normal | 0 | 0 |
| | Conjunctivae | Normal | 0 | 0 |
| | Chemosis | Normal | 0 | 0 |
| 48 h | Cornea | No ulceration | 0 | 0 |
| | | or opacity | | |
| | Iris | Normal | 0 | 0 |
| | Conjunctivae | Normal | 0 | 0 |
| | Chemosis | Normal | 0 | 0 |
| 72 h | Cornea | No ulceration | 0 | 0 |
| | | or opacity | | |
| | Iris | Normal | 0 | 0 |
| | Conjunctivae | Normal | 0 | 0 |
| | Chemosis | Normal | 0 | 0 |

### Confirmatory Test

TABLE 20
Grading for Acute Ocular Irritation
of ML1 A and ML1 B as per OECD TG 405

| | | | Gradings | | | |
|---|---|---|---|---|---|---|
| Time | | | ML1A | | ML1B | |
| Points | Parameters | Observations | 1 | 2 | 1 | 2 |
| 1 h | Cornea | No ulceration or opacity | 0 | 0 | 0 | 0 |
| | Iris | Normal | 0 | 0 | 0 | 0 |
| | Conjunctivae | Normal | 0 | 0 | 0 | 0 |
| | Chemosis | Normal | 0 | 0 | 0 | 0 |
| 24 h | Cornea | No ulceration or opacity | 0 | 0 | 0 | 0 |
| | Iris | Normal | 0 | 0 | 0 | 0 |
| | Conjunctivae | Normal | 0 | 0 | 0 | 0 |
| | Chemosis | Normal | 0 | 0 | 0 | 0 |
| 48 h | Cornea | No ulceration or opacity | 0 | 0 | 0 | 0 |
| | Iris | Normal | 0 | 0 | 0 | 0 |
| | Conjunctivae | Normal | 0 | 0 | 0 | 0 |
| | Chemosis | Normal | 0 | 0 | 0 | 0 |
| 72 h | Cornea | No ulceration or opacity | 0 | 0 | 0 | 0 |
| | Iris | Normal | 0 | 0 | 0 | 0 |
| | Conjunctivae | Normal | 0 | 0 | 0 | 0 |
| | Chemosis | Normal | 0 | 0 | 0 | 0 |

### Results:

1. Both the test solutions were found to be safe for the eye as evident by the absence of any ocular lesions till 72 h.
2. The same finding was further confirmed by the use of 2 additional animals per test solution.
3. The observations were made at 1 h, 24 h, 48 h and 72 h post instillation.
4. No ocular lesion or irritation was observed at any time point during the confirmatory test.

### Conclusion:

Hence, it is concluded that ML1 A and ML1 B are safe for ophthalmic use without any signs of irritation.

### Example 11

The efficacy (intraocular pressure reduction effect) of Brinzolamide solution 1% w/v and 1.25% w/v

Effect of Brinzolamide on α-chymotrypsin induced glaucoma in New Zealand Rabbit was studied. The objective of the study was to induce chronic ocular glaucoma in New Zealand Rabbit by the administration of α-chymotrypsin and to evaluate the effect of Brinzolamide on intraocular pressure (IOP) reduction at specific time intervals.
Animals were divided into Five groups of six animals each
Group I: Normal Control Group (Healthy Volunteer)
Group II: Glaucoma Control Group
Group III: Standard Group (RLD product, Azopt® Brinzolamide 1% suspension)
Group IV: ML1A (Brinzolamide Ophthalmic Solution 1.0% w/v)
Group V: ML1B (Brinzolamide Ophthalmic Solution 1.25% w/v)

### Induction of Chronic Ocular Glaucoma

Two drops of Proparacaine HCl (0.5%) were administered into the left eye of rabbits belonging to Groups II, III, IV and V, followed by the administration of 0.05-0.1 ml of Lignocaine, a local anesthetic into the eye.
Chronic ocular glaucoma was induced by the administration of 0.1 ml of a-chymotrypsin solution (10 mg/ml) prepared in sterile isotonic saline.
After 2-3 days of glaucoma induction, two drops of ciprofloxacin eye drops were instilled in order to avoid topical inflammation
A daily ocular examination was performed for seven days, after which IOP was stabilized and the IOP (zero time) was noted
IOP was recorded thrice per eye and the mean was noted at each time point

### Product Instillation

On IOP stabilization, two-drops of test and standard formulations were instilled into the left eye of rabbits of the respective groups followed by closure of the eyelids for 10 seconds to avoid loss

### Experimental Observations:

IOP was measured with the calibrated Schiotz Tonometer at specific time intervals following TCA (Test Chemical Application)
The change in IOP was calculated using the formula,

$$Δ IOP = IOP zero time - IOP time t$$

IOP zero time means Stabilized IOP on 7th day post induction
ΔIOP is represented as mean (±SEM) of six animals (n=6) for each treatment at each time point
The ΔIOP vs time graph is plotted for each treatment group.
Imax, Tmax and AUC were determined from the plotted graphs.
Imax=Imax−maximum reduction in the intra ocular pressure or Peak decrease in IOP; Tmax=or Time to reach peak decrease in IOP (the time to reach maximum IOP lowering effect after instillation); and AUC=Area Under the ΔIOP vs Time curve

### Statistical Analysis:

Calculation of mean, SEM was done in Microsoft Excel 2021 and AUC determination was done using GraphPad Prism (Version 5.00).

TABLE 21
Effect of Brinzolamide on α-chymotrypsin induced glaucoma in New Zealand Rabbits.

| Time | ΔIOP (mmHg) | | | | |
|---|---|---|---|---|---|
| (h) | Normal control | Glaucoma Control | Standard Group | ML1A | ML1B |
| 0 | 0.000 ± 0.000 | 0.000 ± 0.000 | 0.000 ± 0.000 | 0.000 ± 0.000 | 0.000 ± 0.000 |
| 0.5 | 0.183 ± 0.101 | 0.066 ± 0.183 | 1.416 ± 0.309** | 1.116 ± 0.237 | 2.950 ± 0.609 |
| 1 | 0.166 ± 0.033 | 0.05 ± 0.138 | 3.666 ± 0.470*** | 3.966 ± 0.584*** | 4.466 ± 0.270*** |
| 2 | 0.050 ± 0.061 | −0.033 ± 0.117 | 5.050 ± 0.446*** | 4.650 ± 0.414*** | 5.700 ± 0.485*** |
| 4 | 0.166 ± 0.033 | −0.033 ± 0.125 | 4.916 ± 0.227*** | 6.666 ± 0.530*** | 6.100 ± 0.450*** |
| 6 | 0.016 ± 0.016 | −0.066 ± 0.098 | 4.233 ± 0.264*** | 3.266 ± 0.444*** | 4.066 ± 0.326*** |
| 12 | 0.016 ± 0.016 | −0.166 ± 0.102 | 1.983 ± 0.291*** | 2.583 ± 0.282*** | 3.066 ± 0.256*** |
| 24 | 0.083 ± 0.079 | −0.100 ± 0.122 | 1.316 ± 0.378** | 2.183 ± 0.363*** | 2.183 ± 0.364*** |
| 36 | 0.166 ± 0.055 | −0.133 ± 0.130 | 0.600 ± 0.159 | 1.900 ± 0.305*** | 1.616 ± 0.320*** |
| 48 | 0.116 ± 0.070 | −0.016 ± 0.157 | 0.183 ± 0.090 | 1.350 ± 0.283** | 1.083 ± 0.218*** |

Data represented as mean ± SEM (n = 6) and analysed by two-way ANOVA followed by Bonferroni's post-test.
***p < 0.001 as compared to Glaucoma control group.
**p < 0.01 as compared to Glaucoma control group.

### Results

TABLE 22
Effect of Brinzolamide on α-chymotrypsin
induced glaucoma in New Zealand Rabbits.

| | Imax | Tmax | AUC |
|---|---|---|---|
| | (mmHg) | (h) | (h mmHg) |
| Standard (RLD Product) 1.0% | 5.05 | 2.00 | 79.75 |
| ML1A (Brinzolamide Ophthalmic | 6.66 | 4.00 | 117.30 |
| Solution, 1.0% w/v) | | | |
| ML1B (Brinzolamide Ophthalmic | 6.10 | 4.00 | 121.50 |
| Solution, 1.25% w/v) | | | |

### CONCLUSION

1) ML1A (Brinzolamide Ophthalmic Solution, 1.0% w/v) and ML1B (Brinzolamide Ophthalmic Solution, 1.25% w/v) were found to be safe for ophthalmic use without any sign of irritation or ocular lesion in initial as well as confirmatory tests.
2) The standard as well as test samples (ML1A &ML1B) could significantly reduce the elevated IOP as compared to the glaucoma control group.
3) The extent of IOP reduction for test formulations was greater as compared to the standard treatment exhibiting higher Imax value.
4) The drug release from formulation was found to be sustained as compared to the standard drug exhibiting higher Tmax.
5) The test formulations were found to be more efficacious than the standard as evident from higher AUC values of test samples.

### Example 12

### Effect of Brinzolamide Formulations on Ocular Irritation Test in Rabbits

The objective of the study was to evaluate the Acute Ocular Irritation of ML2A (Brinzolamide 0.5%) and ML2B (Brinzolamide 0.75%) in New Zealand Rabbit

### A) Acute Ocular Irritation of ML2A and ML2B in New Zealand Rabbit.

Acute ocular irritation test was conducted in order to assess the ocular tolerance and toxicity potential of the test formulations. Acute Ocular Irritation test was conducted according to the OECD TG 405. The acute ocular irritation test was performed on healthy New Zealand White Rabbits weighing between 1-3 kg and divided into two groups of ML2 A and ML2 B.

24 h prior to the test chemical application (TCA), animals were observed for any pre-existing eyeirritation, ocular defects or injury. The animals were used for the test only after ensuring the absence on these signs. In the initial test, 1 animal per group was used for the testing. 1 h before the TCA, 0.01 mg/kg buprenorphine hydrochloride (0.3 mg/ml), a systemic analgesic was administered by subcutaneous route to both the animals. 5 min prior to the TCA, 2 drops of Proparacaine HCl (0.5%), a topical anesthetic were instilled to both the eyes of each rabbit. The eye that received topical anesthetic but did not receive the test substance, served as the control. 2 drops of ML2A and ML2B were instilled to the respective rabbits, into the conjunctival sac by gently pulling the lower eyelid and then the lids were gently held together for 10 seconds to prevent the loss of test substance. Eight hours after TCA, 0.01 mg/kg buprenorphine HCl (0.3 mg/ml) and 0.5 mg/kg Meloxicam (0.05 mg/ml) were administered by subcutaneous route. The observations were made for 3 consecutive days at specific time intervals for ocular lesions of cornea, iris, conjunctivae and chemosis and scoring was given according to OECD TG 405.

The ocular safety of the test formulations was confirmed by a confirmatory test using two additional animals for each test sample.

TABLE 23
Initial test
Grading for Acute Ocular Irritation of ML2 A and
ML2 B as per OECD TG 405

| | |
|---|---|
| Time | Gradings |

| Points | Parameters | Observations | ML2A | ML2 B |
|---|---|---|---|---|
| 1 h | Cornea | No ulceration | 0 | 0 |
| | | or opacity | | |
| | Iris | Normal | 0 | 0 |
| | Conjunctivae | Normal | 0 | 0 |
| | Chemosis | Normal | 0 | 0 |
| 24 h | Cornea | No ulceration | 0 | 0 |
| | | or opacity | | |
| | Iris | Normal | 0 | 0 |
| | Conjunctivae | Normal | 0 | 0 |
| | Chemosis | Normal | 0 | 0 |
| 48 h | Cornea | No ulceration | 0 | 0 |
| | | or opacity | | |
| | Iris | Normal | 0 | 0 |
| | Conjunctivae | Normal | 0 | 0 |
| | Chemosis | Normal | 0 | 0 |
| 72 h | Cornea | No ulceration | 0 | 0 |
| | | or opacity | | |
| | Iris | Normal | 0 | 0 |
| | Conjunctivae | Normal | 0 | 0 |
| | Chemosis | Normal | 0 | 0 |

TABLE 24
Confirmatory test
Grading for Acute Ocular Irritation of ML2 A
and ML2 B as per OECD TG 405

| | | | Gradings | | | |
|---|---|---|---|---|---|---|
| Time | | | ML2 A | | ML2 B | |
| Points | Parameters | Observations | 1 | 2 | 1 | 2 |
| 1 h | Cornea | No ulceration or opacity | 0 | 0 | 0 | 0 |
| | Iris | Normal | 0 | 0 | 0 | 0 |
| | Conjunctivae | Normal | 0 | 0 | 0 | 0 |
| | Chemosis | Normal | 0 | 0 | 0 | 0 |
| 24 h | Cornea | No ulceration or opacity | 0 | 0 | 0 | 0 |
| | Iris | Normal | 0 | 0 | 0 | 0 |
| | Conjunctivae | Normal | 0 | 0 | 0 | 0 |
| | Chemosis | Normal | 0 | 0 | 0 | 0 |
| 48 h | Cornea | No ulceration or opacity | 0 | 0 | 0 | 0 |
| | Iris | Normal | 0 | 0 | 0 | 0 |
| | Conjunctivae | Normal | 0 | 0 | 0 | 0 |
| | Chemosis | Normal | 0 | 0 | 0 | 0 |
| 72 h | Cornea | No ulceration or opacity | 0 | 0 | 0 | 0 |
| | Iris | Normal | 0 | 0 | 0 | 0 |
| | Conjunctivae | Normal | 0 | 0 | 0 | 0 |
| | Chemosis | Normal | 0 | 0 | 0 | 0 |

Conclusion:
ML2A and ML2B were found to be safe for ophthalmic use without any sign of irritation or ocular lesion in initial as well as confirmatory tests.

### Example 13

### The Efficacy (Intraocular Pressure Reduction Effect) of Brinzolamide Solution 1% w/v and 1.25% w/v

Effect of Brinzolamide on a-chymotrypsin induced glaucoma in New Zealand Rabbit was studied. The objective of the study was to induce chronic ocular glaucoma in New Zealand Rabbit by the administration of a-chymotrypsin and to evaluate the effect of Brinzolamide on intraocular pressure (IOP) reduction at specific time intervals.

To study the effect of Brinzolamide, animals were divided into 4 groups of 12 animals each.
Group I: Normal Control Group (Healthy Volunteer)
Group II: Glaucoma Control Group
Group III: Test Group ML2 A (Brinzolamide Ophthalmic Solution 0.5% w/v)
Group IV: Test Group ML2 B (Brinzolamide Ophthalmic Solution 0.75% w/v)

### Induction of Chronic Ocular Glaucoma:

Induction procedure began with the administration of 2 drops of Proparacaine HCl (0.5%) into the left eye of rabbits belonging to Groups II, III and IV. This was followed by administration of 0.05-0.1 ml of Lignocaine, a local anaesthetic into the eye.
Chronic ocular glaucoma was induced by the administration of 0.1 ml of α-chymotrypsin solution (10 mg/ml) prepared in sterile isotonic saline.
After 2-3 days of Glaucoma induction, 2 drops of ciprofloxacin eye drops were instilled in order to avoid topical inflammation.
A daily ocular examination was performed for 7 days, after which IOP was stabilized and the IOP (zero time) was noted.
IOP was recorded thrice per eye and the mean was noted at each time point.

### Product Instillation

On IOP stabilization, 2 drops of test and standard formulations were instilled into the left eye of rabbits of the respective groups followed by closure of the eyelids for 10 seconds to avoid loss.

### Experimental Observations:

IOP was measured with the calibrated Schiotz tonometer.
The change in IOP from the stabilized IOP was calculated by using the formula:

$$Δ IOP = IOP zero time - IOP time t$$

IOP zero time means Stabilized IOP on 7th day post induction
ΔIOP is represented as mean (±SEM) of twelve animals (n=12) for each treatment at each time point.
The ΔIOP vs time graph is plotted for each treatment group.
Iₘₐₓ, Tₘₐₓ and AUC were determined from the plotted graphs, on which basis the different formulations were compared.

TABLE 25
Effect of Brinzolamide on α-chymotrypsin
induced glaucoma in New Zealand Rabbit.

| Time | ΔIOP (mmHg) | | | |
|---|---|---|---|---|
| (h) | Normal control | Glaucoma Control | ML2 A | ML2 B |
| 0 | 0 ± 0 | 0 ± 0 | 0 ± 0 | 0 ± 0 |
| 0.5 | −0.008 ± 0.071 | −0.008 ± 0.079 | 0.891 ± 0.137** | 1.200 ± 0.209*** |
| 1 | 0.000 ± 0.075 | −0.016 ± 0.099 | 2.108 ± 0.271*** | 2.566 ± 0.298*** |
| 2 | 0.091 ± 0.054 | −0.091 ± 0.112 | 3.366 ± 0.299*** | 3.783 ± 0.278*** |
| 4 | 0.141 ± 0.052 | 0.050 ± 0.107 | 3.116 ± 0.261*** | 4.208 ± 0.270*** |
| 6 | 0.158 ± 0.089 | 0.000 ± 0.077 | 2.508 ± 0.264*** | 3.225 ± 0.225*** |
| 12 | 0.100 ± 0.077 | 0.016 ± 0.107 | 1.633 ± 0.243*** | 2.108 ± 0.189*** |
| 24 | 0.100 ± 0.072 | 0.058 ± 0.091 | 1.108 ± 0.214*** | 1.691 ± 0.238*** |
| 36 | 0.058 ± 0.083 | 0.158 ± 0.074 | 0.800 ± 0.138 | 0.983 ± 0.209** |
| 48 | 0.050 ± 0.080 | −0.05 ± 0.106 | 0.216 ± 0.091 | 0.341 ± 0.158 |

Data represented as mean ± SEM (n = 12) and analysed by two-way ANOVA followed by Bonferroni's post-test.
***p < 0.001 as compared to Glaucoma control group.
**p < 0.01 as compared to Glaucoma control group.

TABLE 26
Effect of Brinzolamide on α-chymotrypsin
induced glaucoma in New Zealand Rabbit.

| | Imax (mmHg) | Tmax (h) | AUC (h mmHg) |
|---|---|---|---|
| ML2A | 3.366 | 2 | 62.24 |
| ML2B | 4.208 | 4 | 82.64 |

### Statistical Analysis:

Calculation of mean, SEM was done in Microsoft Excel 2021. AUC determination was done using GraphPad Prism (Version 5.00).

### Analysis:

After 7 days of α-chymotrypsin induction, the IOP was found to be higher than the normal IOP range (10-21 mmHg). Respective treatments were given to the groups on 7th day. In the normal control group, the IOP ranged between 15-20 mmHg without much variation throughout the treatment period. In the glaucoma control group, the IOP was stabilized to 28.64 mmHg 7 days post induction. The IOP was stabilized at 29.25 mmHg 7 days post induction in the ML2 A treatment group. After TCA, Tmax was observed at 2 h with maximum IOP reduction value of 3.366 mmHg as compared to the stabilized IOP and AUC of 62.24 h mmHg in ML2 A treated group. In the ML2 B treated group, IOP was stabilized to 28.46 mmHg, which was found to reduce following the treatment with ML2 B. The peak decrease in IOP was found at 4 h with maximum IOP reduction of 4.208 mmHg and covering the AUC of 82.64 h mmHg. The AUC and IOP reduction of ML2 A and ML2 B treated groups was significantly higher as compared to the glaucoma control group which shows the high efficacy of test samples in reducing the IOP.

### Conclusion:

ML2A and ML2B were found to be safe for ophthalmic use without any sign of irritation or ocular lesion in initial as well as confirmatory tests.
The test samples could significantly reduce the elevated IOP as compared to the glaucoma control group.
The extent of IOP reduction for test formulations was greater as compared to the glaucoma control group exhibiting higher Imax value.
The drug release from formulation was found to be sustained for ML2B as compared to the ML2A exhibiting higher Tmax.
ML2B was found to be more efficacious than ML2 A as evident from higher AUC values of ML2B.

We claim:

1. An aqueous micelle solution comprising Brinzolamide dispersed uniformly in a polymer; in the form of polymeric micelles; and one or more pharmaceutically acceptable excipients; wherein the polymer in the micelle is a polyvinyl caprolactam-polyvinyl acetate-polyethylene glycol graft co-polymer; and wherein the aqueous micelle solution does not contain a surfactant.

2. The aqueous micelle solution as claimed in claim 1 comprises 1% w/v Brinzolamide, wherein the solution lowers intraocular pressure in a mammal by at least 4 mm Hg within 2 hours upon instillation into the eye of the mammal.

3. The aqueous micelle solution as claimed in claim 1 comprises 1.25% w/v Brinzolamide, wherein the solution lowers intraocular pressure in a mammal by at least 5 mm Hg within 2 hours upon instillation into the eye of the mammal.

4. The aqueous micelle solution as claimed in claim 1 comprises 0.75% w/v Brinzolamide, wherein the solution lowers intraocular pressure in a mammal by at least 3.5 mm Hg within 2 hours upon instillation into the eye of the mammal.

5. The aqueous micelle solution as claimed in claim 1 comprises 0.5% w/v Brinzolamide; wherein the solution lowers intraocular pressure in a mammal by at least 3 mm Hg within 2 hours upon instillation into the eye of the mammal.

6. The aqueous micelle solution as claimed in claim 2, wherein the Brinzolamide is present in an amount of 1% w/v of the solution and the Tmax is reached between 3.5 to 4.5 hours.

7. The aqueous micelle solution as claimed in claim 2, wherein the Brinzolamide is present in an amount of 1% w/v of the solution and the AUC is about 117 h mm Hg.

8. The aqueous micelle solution as claimed in claim 2, wherein the Brinzolamide is present in an amount of 1% w/v of the solution and the Tmax is reached at about 4 hours.

9. The aqueous micelle solution as claimed in claim 3, wherein the Brinzolamide is present in an amount of 1.25% w/v of the solution and the Tmax is reached at about 4 hours.

10. The aqueous micelle solution as claimed in claim 1, wherein the polymeric micelles have a size with D₉₀ in a range from about 130 nm to 200 nm.

11. The aqueous micelle solution as claimed in claim 1, wherein the aqueous micelle solution is stable at 40° C. and 75% relative humidity for 6 months.

12. A method of preparing the aqueous micelle solution as claimed in claim 1, comprising the step of: preparing solid dispersions of Brinzolamide and polyvinyl caprolactam-polyvinyl acetate-polyethylene glycol graft co-polymer by homogeneous mixing of Brinzolamide and polyvinyl caprolactam-polyvinyl acetate-polyethylene glycol graft copolymer; and mixing one or more pharmaceutically acceptable excipients and water with the solid dispersions, wherein the pharmaceutically acceptable excipients does not contain a surfactant.

13. The method as claimed in claim 12, wherein the solid dispersions are prepared by a solvent evaporation method, or by a hot melt extrusion method.

14. The method as claimed in claim 12, wherein the weight ratio of the Brinzolamide to the polyvinyl caprolactam-polyvinyl acetate-polyethylene glycol graft co-polymer in the solid dispersion is within the range of 1:2.5 to 1:15.

15. The method as claimed in claim 12, wherein the weight ratio of the Brinzolamide to the polyvinyl caprolactam-polyvinyl acetate-polyethylene glycol graft co-polymer in the solid dispersion is within the range of 1:10 to 1:15.
